SEQUENCE LISTING (20 sequences)

SEQ ID NO: 1 - protein, 612 aa, Musca domestica
MQKMDNLEVKAKINQGLYKITPRHKGTSFIWNVLADIQKEDDTLVEGWVFCRKCEKVLKYTTRQTSNLCRHKCCASLKQSRELKTVSADCKKEAIEKCAQWVVRDCRPFSAVSGSGFIDMIKFFIKVGAEYGEHVNVEELLPSPITLSRKVTSDAKEKKALISREIKSAVEKDGASATIDLWTDNYIKRNFLGVTLHYHENNELRDLILGLKSLDFERSTAENIYKKLKAIFLQFNVEDLSSIKFVTDRGANVVKSLANNIRINCSSHLLSNVLENSFEETPELNVPILACKNIVKYFKKANLQHRLRSSLKSECPTRWNSTYTMLRSILDNWESVIQILSEAGETQRIVHINKSIIQTMVNILDGFERIFKELQTCSSPSLCFVVPSILKVKEICSPDVGDVADIAKLKVNIIKNVRIIWEENLSIWHYTAFFFYPPALHMQQEKVAQIKEFCLSKMEDLELINRMSSFNELSATQLNQSDSNSHNSIDLTSHSKDISTTSFFFPQLTQNNSREPPVCPSDEFEFYRKEIVILSEDFKVMEWWNLNSKKYPKLSKLALSLLSIPASSAASERTFSLAGNIITEKRNRIGQQTVDSLLFLNSFYKNFCKLDI

SEQ ID NO: 2 - DNA, 1839 bp, Musca domestica
atgcagaaaatggacaatttggaagtgaaagcaaaaatcaaccaaggattatataaaattactccgcgacataaaggaacaagttttatttggaacgttttagcggatatacagaaagaagacgatacattggtggaagggtgggtgttttgccgaaaatgcgaaaaagttttaaaatacacaactaggcagacatcaaacttatgtcgtcataaatgctgtgcctctctaaagcaatcccgagaattaaaaactgtttcagctgattgcaaaaaggaagcaattgaaaaatgtgcacaatgggtggtacgagattgtcggcctttttcggccgtctctggatccggctttatcgatatgataaaattttttattaaagttggagccgaatatggtgaacatgtcaacgttgaggaattgttaccaagtccaataacgctatcgagaaaggtaacttcggatgcaaaagaaaaaaaagctctgattagtcgagaaattaagtctgctgtagagaaagatggtgcatcagcaacgatagatttgtggaccgataattatataaaacggaattttttgggagtaacgttacactaccatgaaaacaatgaactgcgagatctaattttaggtttaaagtccttagattttgaaagatccacagcagaaaatatttataagaagcttaaagccatttttttacaattcaacgtcgaagacttgagtagtataaaatttgtgacagatagaggagccaatgtcgtaaaatcattggcaaataatatcagaattaactgcagcagccatttgctttcaaacgtgttggaaaattcatttgaggagacacctgaactcaatgtgcctattcttgcttgcaaaaatattgtaaaatatttcaagaaagccaatctgcagcacagacttcgaagttctttaaaaagtgagtgccctacacggtggaattccacatacacgatgcttcgatctattctcgacaactgggaaagcgtgattcaaatattaagtgaggcgggagagacacagagaattgttcatataaataagtcgataattcaaacaatggtcaacatcctcgatgggtttgaaagaatttttaaagaattacaaacatgcagttcaccatctctgtgttttgttgtgccttccattttaaaagtaaaagaaatatgttcacctgacgttggcgacgttgcagatatagcaaaattgaaagtgaacattataaaaaatgtaagaataatatgggaagaaaatttaagcatatggcactacacagcattttttttctatccgcccgccttgcatatgcaacaagagaaagtggcacaaattaaagaattttgcttatccaaaatggaagatttggaattaataaaccgcatgagttcctttaacgaattatccgcaactcagcttaaccagtcggactccaatagccacaacagtatagatttaacatcccattcaaaagacatttcaacgacaagtttctttttcccgcaattaactcagaacaatagtcgtgagccaccagtgtgtccaagcgatgaatttgaattttatcgtaaagaaatagttattttaagcgaagattttaaagttatggaatggtggaatcttaattcaaaaaagtatcctaaactatctaaactggctttgtcgttattatcaatacctgcaagtagcgctgcatcggaaaggacattttccctagctggaaatataataactgaaaagagaaacaggattgggcaacaaactgtcgacagcttgttatttttaaattccttttacaaaaatttttgtaaattagatatataa

SEQ ID NO: 3 - DNA, 17 bp, Musca domestica
cttgttgttgttctctg

SEQ ID NO: 4 - DNA, 17 bp, Musca domestica
cttgttgaagttctctg

SEQ ID NO: 5 - protein, 612 aa, Musca domestica
MEKMDNLEVKAKINQGLYKITPRHKGTSFIWNVLADIQKEDDTLVEGWVFCRKCEKVLKYTTRQTSNLCRHKCCASLKQSRELKTVSADCKKEAIEKCAQWVVRDCRPFSAVSGSGFIDMIKFFIKVGAEYGEHVNVEELLPSPITLSRKVTSDAKEKKALISREIKSAVEKDGASATIDLWTDNYIKRNFLGVTLHYHENNELRDLILGLKSLDFERSTAENIYKKLKAIFSQFNVEDLSSIKFVTDRGANVVKSLANNIRINCSSHLLSNVLENSFEETPELNMPILACKNIVKYFKKANLQHRLRSSLKSECPTRWNSTYTMLRSILDNWESVIQILSEAGETQRIVHINKSIIQTMVNILDGFEAIFKELQTCSSPSLCFVVPSILKVKEICSPDVGDVADIAKLKVNIIKNVRIIWEENLSIWHYTAFFFYPPALHMQQEKVAQIKEFCLSKMEDLELINRMSSFNELSATQLNQSDSNSHNSIDLTSHSKDISTTSAAFPQLTQNNSREPPVCPSDEFEFYRKEIVILSEDFKVMEWWNLNSKKYPKLSKLALSLLSIPASSAASERTFSLAGNIITEKRNRIGQQTVDSLLFLNSFYKNFCKLDI

SEQ ID NO: 6 - DNA, 1839 bp, Musca domestica
atggagaaaatggacaatttggaagtgaaagcaaaaatcaaccaaggattatataaaattactccgcgacataaaggaacaagttttatttggaacgttttagcggatatacagaaagaagacgatacattggtggaagggtgggtgttttgccgaaaatgcgaaaaagttttaaaatacacaactaggcagacatcaaacttatgtcgtcataaatgctgtgcctctctaaagcaatcccgagaattaaaaactgtttcagctgattgcaaaaaggaagcaattgaaaaatgtgcacaatgggtggtacgagattgtcggcctttttcggccgtctctggatccggctttatcgatatgataaaattttttattaaagttggagccgaatatggtgaacatgtcaacgttgaggaattgttaccaagtccaataacgctatcgagaaaggtaacttcggatgcaaaagaaaaaaaagctctgattagtcgagaaattaagtctgctgtagagaaagatggtgcatcagcaacgatagatttgtggaccgataattatataaaacggaattttttgggagtaacgttacactaccatgaaaacaatgaactgcgagatctaattttaggtttaaagtccttagattttgaaagatccacagcagaaaatatttataagaagcttaaagccattttttcacaattcaacgtcgaagacttgagtagtataaaatttgtgacagatagaggagccaatgtcgtaaaatcattggcaaataatatcagaattaactgcagcagccatttgctttcaaacgtgttggaaaattcatttgaggagacacctgaactcaatatgcctattcttgcttgcaaaaatattgtaaaatatttcaagaaagccaatctgcagcacagacttcgaagttctttaaaaagtgagtgccctacacggtggaattccacatacacgatgcttcgatctattctcgacaactgggaaagcgtgattcaaatattaagtgaggcgggagagacacagagaattgttcatataaataagtcgataattcaaacaatggtcaacatcctcgatgggtttgaagcaatttttaaagaattacaaacatgcagttcaccatctctgtgttttgttgtgccttccattttaaaagtaaaagaaatatgttcacctgacgttggcgacgttgcagatatagcaaaattgaaagtgaacattataaaaaatgtaagaataatatgggaagaaaatttaagcatatggcactacacagcattttttttctatccgcccgccttgcatatgcaacaagagaaagtggcacaaattaaagaattttgcttatccaaaatggaagatttggaattaataaaccgcatgagttcctttaacgaattatccgcaactcagcttaaccagtcggactccaatagccacaacagtatagatttaacatcccattcaaaagacatttcaacgacaagtgccgctttcccgcaattaactcagaacaatagtcgtgagccaccagtgtgtccaagcgatgaatttgaattttatcgtaaagaaatagttattttaagcgaagattttaaagttatggaatggtggaatcttaattcaaaaaagtatcctaaactatctaaactggctttgtcgttattatcaatacctgcaagtagcgctgcatcggaaaggacattttccctagctggaaatataataactgaaaagagaaacaggattgggcaacaaactgtcgacagcttgttatttttaaattccttttacaaaaatttttgtaaattagatatatag

SEQ ID NO: 7 - protein, 592 aa, Musca domestica
MEKMDNLEVKAKINQGLYKITPRHKGTSFIWNVLADIQKEDDTLVEGWVFCRKCEKVLKYTTRQTSNLCRHKCCASLKQSRELKTVSADCKKEAIEKCAQWVVRDCRPFSAVSGSGFIDMIKFFIKVGAEYGEHVNVEELLPSPITLSRKVTSDAKEKKALISREIKSAVEKDGASATIDLWTDNYIKRNFLGVTLHYHENNELRDLILGLKSLDFERSTAENIYKKLKAIFSQFNVEDLSSIKFVTDRGANVVKSLANNIRINCSSHLLSNVLENSFEETPELNMPILACKNIVKYFKKANLQHRLRSSLKSECPTRWNSTYTMLRSILDNWESVIQILSEAGETQRIVHINKSIIQTMVNILDGFERIFKELQTCSSPSLCFVVPSILKVKEICSPDVGDVADIAKLKVNIIKNVRIIWEENLSIWHYTAFFFYPPALHMQQEKVAQIKEFCLSKMEDLELINRMSSFNELSATQLNQSDSNSHNSIDLTSHSKPVCPSDEFEFYRKEIVILSEDFKVMEWWNLNSKKYPKLSKLALSLLSIPASSAASERTFSLAGNIITEKRNRIGQQTVDSLLFLNSFYKNFCKLDI

SEQ ID NO: 8 - DNA, 1779 bp, Musca domestica
atggagaaaatggacaatttggaagtgaaagcaaaaatcaaccaaggattatataaaattactccgcgacataaaggaacaagttttatttggaacgttttagcggatatacagaaagaagacgatacattggtggaagggtgggtgttttgccgaaaatgcgaaaaagttttaaaatacacaactaggcagacatcaaacttatgtcgtcataaatgctgtgcctctctaaagcaatcccgagaattaaaaactgtttcagctgattgcaaaaaggaagcaattgaaaaatgtgcacaatgggtggtacgagattgtcggcctttttcggccgtctctggatccggctttatcgatatgataaaattttttattaaagttggagccgaatatggtgaacatgtcaacgttgaggaattgttaccaagtccaataacgctatcgagaaaggtaacttcggatgcaaaagaaaaaaaagctctgattagtcgagaaattaagtctgctgtagagaaagatggtgcatcagcaacgatagatttgtggaccgataattatataaaacggaattttttgggagtaacgttacactaccatgaaaacaatgaactgcgagatctaattttaggtttaaagtccttagattttgaaagatccacagcagaaaatatttataagaagcttaaagccattttttcacaattcaacgtcgaagacttgagtagtataaaatttgtgacagatagaggagccaatgtcgtaaaatcattggcaaataatatcagaattaactgcagcagccatttgctttcaaacgtgttggaaaattcatttgaggagacacctgaactcaatatgcctattcttgcttgcaaaaatattgtaaaatatttcaagaaagccaatctgcagcacagacttcgaagttctttaaaaagtgagtgccctacacggtggaattccacatacacgatgcttcgatctattctcgacaactgggaaagcgtgattcaaatattaagtgaggcgggagagacacagagaattgttcatataaataagtcgataattcaaacaatggtcaacatcctcgatgggtttgaaagaatttttaaagaattacaaacatgcagttcaccatctctgtgttttgttgtgccttccattttaaaagtaaaagaaatatgttcacctgacgttggcgacgttgcagatatagcaaaattgaaagtgaacattataaaaaatgtaagaataatatgggaagaaaatttaagcatatggcactacacagcattttttttctatccgcccgccttgcatatgcaacaagagaaagtggcacaaattaaagaattttgcttatccaaaatggaagatttggaattaataaaccgcatgagttcctttaacgaattatccgcaactcagcttaaccagtcggactccaatagccacaacagtatagatttaacatcccattcaaaaccagtgtgtccaagcgatgaatttgaattttatcgtaaagaaatagttattttaagcgaagattttaaagttatggaatggtggaatcttaattcaaaaaagtatcctaaactatctaaactggctttgtcgttattatcaatacctgcaagtagcgctgcatcggaaaggacattttccctagctggaaatataataactgaaaagagaaacaggattgggcaacaaactgtcgacagcttgttatttttaaattccttttacaaaaatttttgtaaattagatatatag

SEQ ID NO: 9 - DNA, 31 bp, Musca domestica
  misc_feature (1)..(1): 5′ T is phosphorylated
tcagagaacaacaacaagtggcttattttga

SEQ ID NO: 10 - DNA, 30 bp, Musca domestica
tcaaaataagccacttgttgttgttctctg

SEQ ID NO: 11 - DNA, 55 bp, artificial sequence
  synthetic copy of modified Hermes transposon
ataagtagcaagtggcgcataagtatcaaaataagccacttgttgttgttctctg

SEQ ID NO: 12 - DNA, 56 bp, artificial sequence
  derivatizedhermes transponson
ccagagaacaacaacaagtggcttattttgatacttatgcgccacttgctacttat

SEQ ID NO: 13 - DNA, 38 bp, artificial sequence
  linker for adding tags
tagtcccttaagcggagccctatagtgagtcgtattac

SEQ ID NO: 14 - DNA, 36 bp, artificial sequence
  linker for ligating tags
gtaatacgactcactatagggctccgcttaagggac

SEQ ID NO: 15 - DNA, 86 bp, artificial sequence
  proprietary sequence (sequencing primer), 4- bp barcode and the Hermes L-end complementary sequence
aatgatacggcgaccaccgagatctacactctttccctacacgacgctcttccgatctgcgtcgcataagtatcaaaataagccac

SEQ ID NO: 16 - DNA, 56 bp, artificial sequence
  3′ linker/adapter specific primer with  3′ Illumina tag for use with SEQ ID NO15
caagcagaagacggcatacgagctcttccgatctgtaatacgactcactatagggc

SEQ ID NO: 17 - DNA, 92 bp, artificial sequence
  tag A sequencing priming region, 4 bp barcode and a 30 bp Hermes Transposon end (Biotinylated top strand)
aatgatacggcgaccaccgagatctacactctttccctacacgacgctcttccgatctgcgttcaaaataagccacttgttgttgttctctg

SEQ ID NO: 18 - DNA, 93 bp, artificial sequence
  Phosphorylated bottom strand Hermes LE sequence for use with SEQ ID NO17
ccagagaacaacaacaagtggcttattttgaacgcagatcggaagagcgtcgtgtagggaaagagtgtagatctcggtggtcgccgtatcatt

SEQ ID NO: 19 - DNA, 92 bp, artificial sequence
  for introducing tag B
caagcagaagacggcatacgagctcacactctttccctacacgacgctcttccgatctgcgttcaaaataagccacttgttgttgttctctg

SEQ ID NO: 20 - DNA, 93 bp, artificial sequence
  Bottom strand for use with Seq ID No19
ccagagaacaacaacaagtggcttattttgaacgcagatcggaagagcgtcgtgtagggaaagagtgtgagctcgtatgccgtcttctgcttg

(19) United States
(12) Patent Application Publication
(10) Pub. No.: US 2015/0284768 A1
(43) Pub. Date: Oct. 8, 2015

(54) EUKARYOTIC TRANSPOSASE MUTANTS AND TRANSPOSON END COMPOSITIONS FOR MODIFYING NUCLEIC ACIDS AND METHODS FOR PRODUCTION AND USE IN THE GENERATION OF SEQUENCING LIBRARIES

(71) Applicant: The Johns Hopkins University, Baltimore, MD (US)

(72) Inventors: Nancy Craig, Baltimore, MD (US); Fred Dyda, Washington, DC (US); Sunil Gangadharan, Baltimore, MD (US); Alison B. Hickman, Washington, DC (US)

(21) Appl. No.: 14/403,506

(22) PCT Filed: May 29, 2013
(86) PCT No.: PCT/US2013/043138

§ 371 Date: Nov. 24, 2014

Related U.S. Application Data
(60) Provisional application No. 61/652,560, filed on May 29, 2012.

(51) Int. Cl. C12Q 1/68; C12N 9/12

(52) U.S. Cl. CPC C12Q 1/6806; C12N 9/1241

(57) ABSTRACT

Novel hyperactive Hermes Transposase mutants and genes encoding them are disclosed. These transposases are easily purified in large quantity after expression in bacteria. The modified Hermes Transposases are soluble and stable and exist as smaller active complexes compared to the native enzyme. The consensus target DNA recognition sequence is the same as the native enzyme and shows minimal insertional sequence bias. These properties are useful in whole genome sequencing applications that involve sample DNA preparation requiring simultaneous fragmentation and attachment of custom sequences to the ends of the fragments. Methods and compositions using these transposases in fragmentation and 5′ end-tagging are also disclosed.

### CROSS-REFERENCE TO PRIOR APPLICATIONS

This application is based on and claims priority and benefit of U.S. Provisional Patent Application 61/652,560 filed on May 29, 2012 which application is incorporated herein by reference to the extent permitted by applicable laws and regulations.

### U.S. GOVERNMENT SUPPORT

This invention was made with U.S. government support. The U.S. government has certain rights in the invention.

### SEQUENCE LISTING

The instant application contains a Sequence Listing generated by Patent-In v. 3.5 which has been submitted in ASCII format via EFS-Web and is hereby incorporated by reference in its entirety to the extent permitted by applicable statute and regulation. Said ASCII file, created on 24 May 2013, is named P11965_{—}02_ST25.txt and is 27 kilobytes in size.

### FIELD OF THE INVENTION

The current invention relates to mutated transposases and methods to use them for fragmenting and tagging target DNA for use in next generation DNA sequencing.

### DESCRIPTION OF THE BACKGROUND

Transposons, segments of DNA that can mobilize to other locations in a genome, are useful for insertion mutagenesis and for generation of priming sites for sequencing of DNA molecules. In vitro, transpositions using transposases and transposons can be used to generate mutagenized plasmid/fosmid libraries for large scale phenotypic screening. More recently, the ability of transposase and transposon end compositions to bring about fragmentation and 5′ tagging of DNA has been exploited in generating libraries of tagged DNA fragments for Next Generation sequencing platforms. Such applications for “cut and paste” DNA transposons Tn5 and Mu and the advantages of using them over methods involving mechanical fragmentation are disclosed in Published U.S. Patent Application 2011/0287435. For these uses, a transposon with minimal insertion bias is desired to allow complete coverage with minimal oversampling. Tn5 and Mu transposons show unfavorable insertional sequence bias. A modified Tn7 TnsABC-only system has low sequence bias but requires the expression and purification of several different subunits to form the active complex and is therefore cumbersome to exploit commercially. Moreover, the frequency of transposition is very low for most transposons and there is a requirement in the art for hyperactive transposases. The modified Hermes Transposase of the present invention is a substantial improvement for the above mentioned applications because of the combination of its higher activity and reduced insertional bias. Transposons have also been used in vivo in generating transgenic organisms as disclosed in Published U.S. Patent Application 2003/0150007. The modified form of Hermes Transposase can also be used for such in vivo applications. In vivo insertional mutagenesis methods using transposons in general e.g. Hermes is disclosed in Published U.S. Patent Application 2004/0092018. These patent applications are incorporated herein by reference to the extent permitted by applicable statute and regulation.

### BRIEF SUMMARY OF THE INVENTION

The mutant transposases disclosed in this invention are a modified form of the native Hermes Transposase, have a similar mechanism of action as the wild type, can easily be expressed in the bacterium, E. coli, and purified in large quantities. These inventive transposases also have the additional advantage of not requiring a preformed transposase complex as in existing alternative transposons such as Tn5 and Mu.6. The inventive transposases, unlike alternatives that have to be incubated at 37° C., is fully active at room temperature at 23° C. up to 30° C. so that the reaction can be readily carried out on a laboratory benchtop.

The modified Hermes Transposases of the invention, as a result of the introduced mutations form a smaller complex (a dimer rather than the inhibited hexameric/octameric form). These Hermes Transposases also have a higher transposition activity in vitro than do the wild type transposase. Compared to existing commercialized transposases, the inventive modified Hermes Transposases have less insertional sequence bias when used for in vitro fragmentation of genomic DNA and 5′ end tagging followed by next generation sequencing

### DESCRIPTION OF THE FIGURES

FIG. 1 illustrates WT, delta497-516, and Triple mutant polypeptide chains;

FIG. 2 shows the Hermes mechanism including excision and strand transfer;

FIG. 3 shows the modeled quaternary crystal structure of the wild type (WT) Hermes octamer;

FIG. 4 is a diagram showing the relationship between the wild type octamer and the mutated dimer interfaces;

FIG. 5 shows HIS6-peptide derivatized Hermes transposon end based fragmentation and tagging;

FIG. 6 is an agarose gel showing activity comparing WT and delta497-516, and Triple mutant Hermes transposases;

FIG. 7 is a diagram of the strand transfer reaction mediated by transposons;

FIG. 8 shows the a general scheme for transposase-based fragmentation and covalent tag attachment to the 5″ ends of target DNA;

FIG. 9 illustrates fragmentation of target DNA and 5′-tagging using a biotinylated Hermes LE and streptavidin beads;

FIG. 10 illustrates fragmentation and tagging using biotinylated Hermes LE, adding a second tag via a different transposase (piggy Bac) for PCR and high throughput sequencing; and

FIG. 11 illustrates fragmentation and tagging using HIS6 peptide tagged Hermes LE oligonucleotides, purification with Ni NTA beads, DNA polymerase extension and strand displacement and final elution with imidazole.

### DETAILED DESCRIPTION OF THE INVENTION

The following description is provided to enable any person skilled in the art to make and use the invention and sets forth the best modes contemplated by the inventors of carrying out their invention. Various modifications, however, will remain readily apparent to those skilled in the art, since the general principles of the present invention have been defined herein specifically to provide improved embodiments of modified Hermes transposases.

Transposons are mobile genetic elements that are an important source of genetic variation and are useful tools for genome engineering, mutagenesis screens, and vectors for transgenesis including gene therapy.

For example, cell free systems for inter-molecular transposition for DNA sequencing, to create deletions or insertions into genes, and for studying protein domain functions have been developed for Tn7 (1), for Tn5 (2), and for Mu (3).

Hermes is a 2479 by long hAT family DNA transposon element derived from the Maryland strain of the common housefly Musca domestica. Its use in creating transgenic insects was disclosed both in a research publication (4), and in U.S. Pat. No. 5,614,398, which is incorporated herein by reference to the extent permissible under applicable statute and regulation.

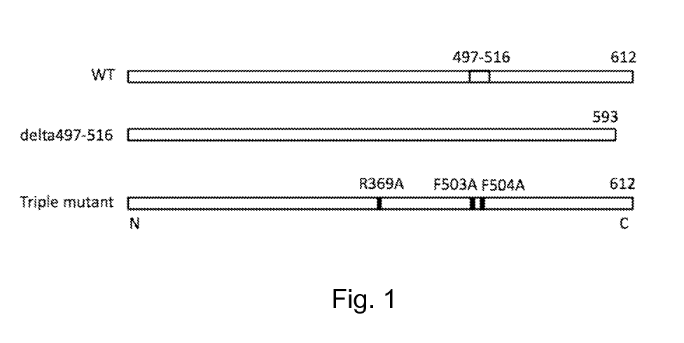

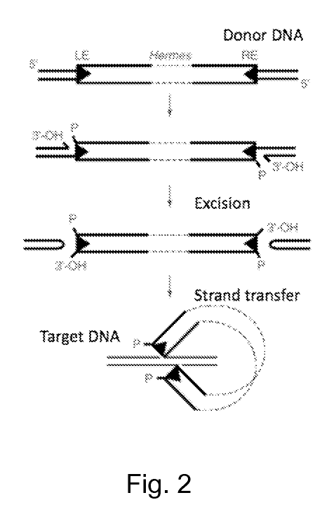

The Hermes transposase gene has since been cloned (SEQ ID NO: 2) and encodes a 612 amino acid polypeptide chain (FIG. 1, SEQ ID NO: 1) similar to other members of the hAT family of transposases, e.g. hobo, Ac and Tam3. The transposon is flanked by 17 by imperfect Left (L-end) and Right (R-end) terminal inverted repeat sequences that are substrates for the transposition reaction (L-end=SEQ ID NO: 3 and R-end=SEQ ID NO: 4) and are similar to other members of the hAT family. Mechanisms involved in Hermes transpositions have been carefully characterized by the inventor N. L. Craig and colleagues. The Hermes Protein facilitates movement of the entire Transposon element by binding initially to each of the two 17 by terminal binding sequences followed by cleavage at both ends of Donor DNA association with target DNA, then, strand transfer and the generation of 8-base-pair (bp) target-site duplications in target DNA upon transposition (5). This scheme is illustrated in FIG. 2 where initial cleavage at the left ends (LE) and right ends (RE) of the Hermes element occurs one nucleotide into the flanking strand of the 5′ ends of the transposon, thereby generating a flanking 3′-OH group. Subsequent nucleophilic attack by this 3′-OH group on the opposite strand results in flanking hairpins and 3′-OH groups at either end of the transposon. These two new 3′-OH groups act as nucleophiles for a coordinated attack on target DNA, in which two insertion events, separated by 8 bp, occur on opposite strands of the Target DNA. This results in addition of lengths of the target DNA onto the transposon effectively inserting the transposon.

The full-length native Hermes transposase (Hermes; residues 1-612) was subcloned into pET-15b (Novagen) for expression in Escherichia coli as an N-terminal His-tag fusion protein and purified. The full-length Hermes transposase (residues 1-612) is soluble, but not readily amenable to crystallization for structural studies because it forms large aggregates in solution when expressed as an N-terminally histidine (His)-tagged fusion protein in E. coli. However, removal of the N-terminal 78 residues results in a version of Hermes that is readily crystallized. The structure of Hermes79-612 was solved using X-ray crystallography (6).

Size-exclusion chromatography and sedimentation equilibrium experiments revealed that Hermes forms multimers in solution and examination of the structure revealed an explanation for the multimerization of Hermes253-612 is provided by the presence of a second interface (interface 2) through which heterodimers can form heterotetramers. This interface arises by domain swapping of two helices between residues 497 and 516 that project away from each Hermes79-612 molecule.

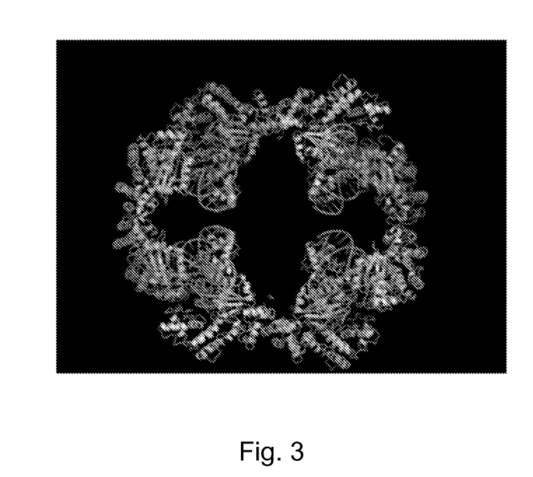

The crystal structure of Hermes79-612 as well as a more recent unpublished structure solved by Alison Hickman and others that reveals the configuration of transposon ends within this structure, see FIG. 3, which made it possible to determine residues in the protein that if mutated or deleted could alter the structure of the multimeric protein complex and its activity (7).

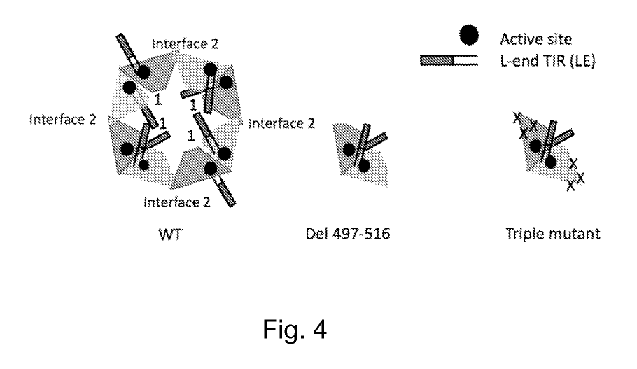

Therefore, several residues were mutated along the polypeptide chain and each mutant tested for its Transposition activity. Two mutants (FIG. 1), the “triple mutant” with a combination of three mutations of residues Arginine to Alanine at position 369, Phenylalanine to Alanine at position 503 and Phenylalanine to Alanine at position 504 in the polypeptide chain (SEQ ID NO: 5 (protein), SEQ ID NO: 6 (nucleic acid)), and the “delta497-516” mutant with a deletion of residues from positions 497 through to position 516 on the polypeptide chain (SEQ ID NO: 7 (protein), SEQ ID NO: 8 (nucleic acid)) formed dimeric complexes in solution and were more active than the native enzyme in in vitro transposition reactions at both 30° C. and at 23° C., using an dsDNA oligonucleotide with the Hermes terminal inverted repeat sequences, a target plasmid, usually pUC19 or pBR322, the purified Hermes transposase and divalent cations such as Mg²⁺ or Mn²⁺. FIG. 4 diagrammatically shows that wild type (WT) Hermes Transposase forms heterodimers which assemble into octamers through the mediation of Interface 2. Both the delta497-516 mutant and the triple mutant lack effective Interface 2s so they form only dimers in solution.

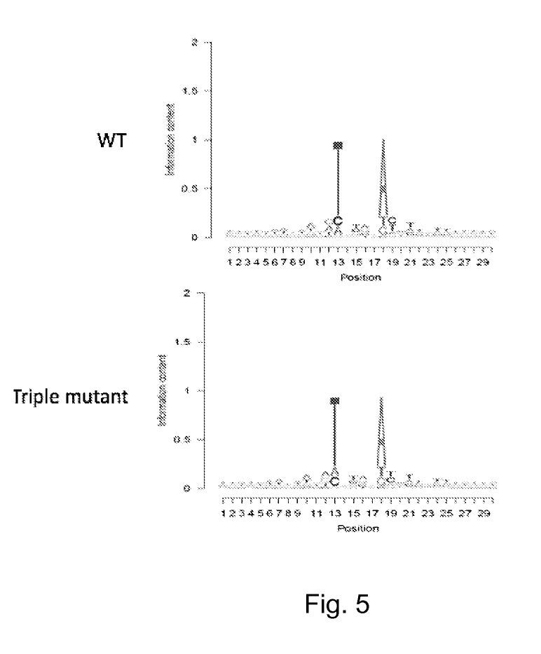

The polypeptide sequences and method of production of the “triple mutant” and the “delta497-516” mutants of Hermes Transposase for in vitro transposition and 5′ tagging of nucleic acids are disclosed herein. Methods of using the above hyperactive forms of the Hermes Transposase in generating genomic 5′ transposon tagged libraries for whole genome amplification and DNA sequencing are also disclosed. The wild type Hermes Transposase showed minimal insertional bias when a very large dataset of in vitro target sites were analyzed by using a standard method (8). Using this approach, in one example where half of a sequencing lane of an Illumina sequencing slide (Illumina, Inc., San Diego, Calif.) was used, 6.5× coverage of the yeast genome was obtained, i.e., on average, each base is contained in 6.5 reads, with only 7.02% of the genome not covered. It was confirmed that the triple mutant did not display any difference in insertional bias. FIG. 5 shows sequence logos of both the wild type (WT) and the triple mutant produced by overlaying the insertion sites of the transposases. The strong thymine and adenine consensus signals indicate essentially no difference in target site selection between the two different transposases.

Methods of purification of hyperactive Hermes Transposase: Method 1. The Hermes transposase (Tnsp) ORF (612 amino acids) was amplified by polymerase chain reaction (PCR) from plasmid pBCHSHH1.9v and cloned between the Ncol and Pvull sites of plasmid pBAD/Myc-HisB (Invitrogen) to generate a Hermes-Myc-His fusion construct, pLQ4. E. coli strain Top10 (Invitrogen) transformed with the Hermes-Myc-His plasmid was grown overnight with shaking at 30° C. in LB medium containing 100 mg/ml carbenicillin. The following day the overnight culture was diluted 1:100 with fresh LB+carbenicillin, and cells were then grown to an absorbance at 600 nm of 0.6 at 30° C. The culture was then shifted to 16° C. and induced with 0.1% L-arabinose for 16 h. After induction, cells were washed by centrifugation at 4° C. with TSG (20 mM Tris-HCl, pH 7.9, 500 mM NaCl, 10% v/v glycerol), and frozen in liquid nitrogen; all subsequent steps were performed at 4° C. Frozen cells were resuspended in 10 ml TSG and lysed by sonication. The cleared lysate was loaded onto a pre-equilibrated Ni²⁺ Sepharose column (Amersham) and washed with ten column volumes of TSG, six column volumes of TSG +50 mM imidazole and six column volumes of TSG+100 mM imidazole. The Hermes-Myc-His fusion protein was eluted with six column volumes of TSG+200 mM imidazole, dialyzed against TSG, and stored at −80° C.

Method 2. Soluble Hermes Transposase (both wild-type and mutants) was obtained by expression in E. coli BL21(DE3) cells which were grown at 310 K until OD600=0.6. Cells were then rapidly cooled on ice to 19° C. and protein expression was induced by addition of IPTG to a final concentration of 0.5 mM. Cells collected from an 8 liter culture were harvested 16-20 h post-induction. The pellet was resuspended in 300 mM NaCl, 12 mM phosphate pH 7.4, flash-frozen in liquid nitrogen and then stored at 193 K. Unless noted otherwise, all purification steps were performed at 4° C. After thawing, cells were lysed by sonication in the presence of 500 mM NaCl, 5 mM imidazole (Im), 25 mM Tris pH 7.5 and 2 mM β-mercaptoethanol (BME). Following centrifugation of the cell lysate at 100,000g for 45 min, the supernatant was loaded onto a Hi-Trap metal-chelation column (Amersham Biosciences) previously equilibrated with NiSO₄. The column was washed extensively with 20 mM Tris pH 7.5, 2 mM Im and 500 mM NaCl followed by the same buffer containing 22 mM Im. Hermes was eluted from the column using a gradient of 22-400 mM Im. After visualization on an SDS-PAGE gel, fractions containing Hermes 79-612 were combined and dialyzed against 20 mM Tris pH 7.5, 1 mM EDTA, 500 mM NaCl, 4 mM BME and 10%(w/v) glycerol. This was followed by dialysis against a single change of the same buffer containing 5 mM dithiothreitol (DTT) in place of BME (TSK buffer). To remove the polyhistidine tag, 10 units of thrombin (Sigma) were added per milligram of protein and incubated overnight. Thrombin was removed by passage over a 1 ml benzamidine Sepharose 4B (Pharmacia) column.

Method 3. Purification of transposase without an affinity tag: It is also possible to purify Hermes transposases in sufficient quantities by expressing a version of the protein that lacks an affinity purification tag. This was done by introducing a stop codon at the position where the sequence corresponding to the tag begins in the Hermes Transposase coding region of pLQ4 of method 1.

Protein was expressed in Top10 cells by growth at 37° C. until OD600 nm ˜0.6, followed by cooling to 19° C. and then induction by addition of arabinose to a final concentration of 0.012%; cells were harvested after 16-18 hrs. Cells were lysed by sonication in Lysis Buffer (25 mM Tris pH 7.5, 0.5 M NaCl, 0.2 mM TCEP), centrifuged to remove cell debris, and the soluble material loaded onto Heparin Sepharose columns (GE Healthcare) previously equilibrated in 25 mM Tris pH 7.5, 0.1 M NaCl, 0.2 mM TCEP. After washing with the same buffer containing 0.5 M NaCl, protein was eluted using a linear gradient from 0.5 M to 1.0 M NaCl. For gel filtration, fractions containing Hermes were combined, concentrated, and loaded onto a preparative scale BioSep-SEC-S 3000 column (Phenomenex) equilibrated in 25 mM HEPES pH 7.3, 1.5 M NaCl, and 0.2 mM TCEP.

Strand transfer assay: Pre-cleaved Hermes-L end for strand-transfer reactions to measure transposition activity was made by annealing the following oligonucleotides:

(top)
(SEQ ID NO: 9)
5′-P-TCAGAGAACAACAACAAGTGGCTTATTTTGA-3′
and
(bottom)
(SEQ ID NO: 10)
5′-TCAAAATAAGCCACTTGTTGTTGTTCTCTG-3′

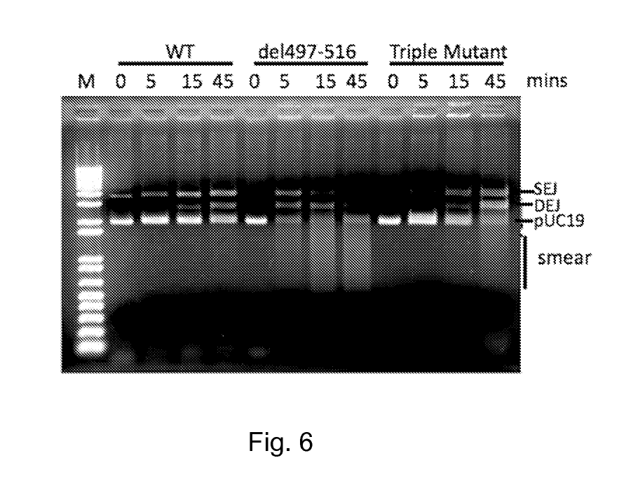

In some experiments, the oligonucleotide was radiolabeled at its 5′ end with y-P³²-dATP (to demonstrate covalent attachment to target) (9 and 10) or, as in the example shown in FIG. 6, unlabeled and used directly as a substrate at 22.9 nM or 60 nM or anywhere from 5 nM to 100 nM for strand-transfer reactions with 3.4 nM or 4 nM pUC19/pBR322 target DNAs and 5 nM to 10.7 nM of Hermes Transposase. In the experiment illustrated in FIG. 6 reactions were incubated for 0 to up to 120 minutes (times of 0, 4, 15, and 45 minutes are shown), at 23° C. or 30° C., preferably 30° C. The reactions were stopped by addition of SDS and EDTA to a concentration of 0.5% -1% SDS and 20-25 mM EDTA, incubated at 65° C. for 20 minutes at room temperature (RT), and in some cases treated with 40 μg of proteinase K and incubated for 30 minutes at 37° C. for analysis. DNA was extracted with phenol/chloroform, precipitated with ethanol and loaded onto 1% TAE agarose gels and/or gel dried and phosphor imaged and the various end products of the reaction analyzed by their distinct electrophoretic mobility. In FIG. 6 the gels were stained with Ethidium bromide to visualize the nucleic acid bands. SEJ and DEJ represent the product of one and two insertions, respectively, per plasmid target molecule. The smear represents the products of fragmentation resulting from more than three insertions per target molecule.

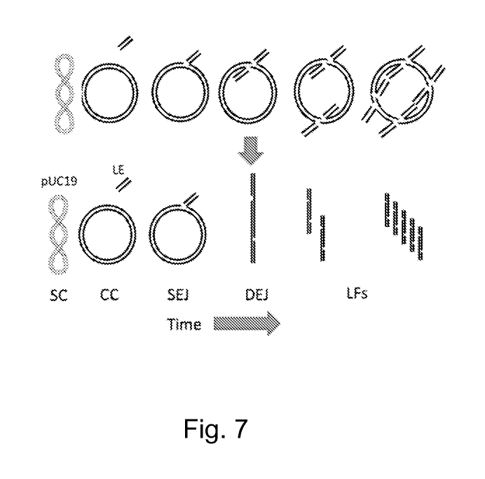

FIG. 7 diagrammatically illustrates the insertion process leading to these results. Transposon Left-end (LE) inserts into supercoiled(SC) plasmid (pUC19) DNA converting it to the nicked circular single end joined (SEJ) configuration and with an additional insertion into the linear double end joined (DEJ) form and with still more insertions into the linear fragments (LFs) that make up the smear.

The dimeric forms of Hermes Transposase are efficient in strand transfer/covalent attachment to target DNA and fragment the target DNA as the reaction proceeds as shown in FIGS. 7 and 6.

Methods of preparing Transposon insertion libraries for high-throughput sequencing.

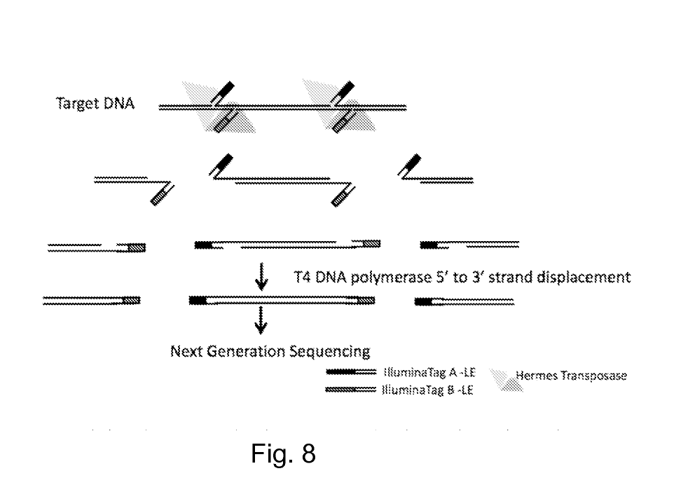

A) Strand transfer reaction: The Strand transfer reaction is diagrammatically illustrated in FIG. 8 where insertion of tagged transposon ends into target DNA results in 8-bp single stranded gaps which are filled in by strand displacing DNA polymerases such as T4 DNA polymerase. This allows Next Gen sequencing platform specific sequences to be attached to fragments of target DNA. Strand transfer reaction was carried out by mixing 285.7 nM (2 ug in 100 uL) purified Hermes transposase, 1 mM (100 pmoles in 100 uL) biotinylated double-stranded Hermes Lend oligonucleotide (LE) containing the 17 bp terminal inverted repeat, prepared by annealing oligonucleotides such as the following:

5′ Biotinylated oligo-Hermes LE Top strand, (SEQ ID No:11) 5′Biotin-ataagtagcaagtggcgcataagtatcaaaataagccaCTTGTTGTTGTTCTCTG and
5′phosphorylated oligo,-Hermes LE Bottom strand, (SEQ ID NO: 12) 5′P-cCAGAGAACAACAACAAGtggcttattttgatacttatgcgccacttgctacttat (Synthesized by IDT) with the addition to 2.53 pM (2 μg in 100 μL) of proteinase K treated-phenol-chloroform purified Schizosaccharomyces pombe or Saccharomyces cerevisae genomic DNA in a buffer containing 25 mM MOPS pH 7.5, 100 mM NaCl, 10 mM MgCl₂, 4% Glycerol, 2 mM DTT, 0.1 mg/mL BSA for 2-3 h at 30° C. The reaction was quenched by adding EDTA and SDS to a final concentration of 20mM and 0.1% respectively and inactivating the enzyme at 65° C. for 20 min. Note that for SEQ ID NO: 11 the uppercase nucleotides represent the 17 bp terminal inverted repeat while the lowercase nucleotides represent the biotin sequencing priming region. For SEQ ID NO: 12 the uppercase nucleotides represent the 17 bp terminal inverted repeat while the lowercase nucleotides represent the sequencing priming region.

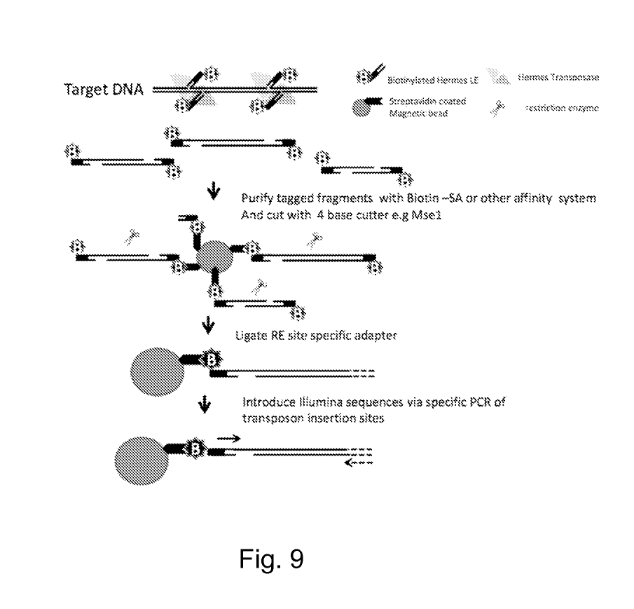

At this stage as shown in FIG. 9, the 3′ end of the top strand of the biotinylated double stranded transposon LE is covalently attached to the 5′ of the target DNA fragment on two ends and fragmentation of the target DNA has occurred along its length. Streptavidin (SA) beads or other affinity systems can be used to purify the tagged fragments. After which the fragments can be cut with a four base cutter such as Mse1. There are several well-known methods for modifying these fragments so that they are prepared as suitable templates for DNA sequencing. For example, specific Next gen sequencing tags such as Illumina sequences can be introduced via specific PCR of the insertion sites. Well-known methods are used to fill in the 8 by gaps in the fragments.

B) Methods of preparing the Transposase mediated Fragmented and 5′ tamed DNA for sequencing: The fragments can, at this stage, be subjected to an extension and strand displacement reaction using DNA polymerase. Arbitrary tags or specific Next gen sequencing platform specific tags (e.g. SEQ ID NOs:17-20) can be added onto the target DNA fragments by this method (see FIG. 9). This method also requires designing primers complementary to the transposon ends in such way that a “suppression PCR” can produce the 5′ (Arbitrary tag A-(LE)) and 3′ (Arbitrary tag B-LE)) Next Gen sequencing tags (as in the Nextera kit, Illumina) on either end of each of the fragments.

Hermes L-end oligo (tag A-LE) with Illumina/arbitrary tag A sequencing priming region, 4 by barcode and a 30 by Hermes Transposon end is prepared by annealing:

tagA-LE top strand (SEQ ID NO: 17): 5′Biotin AATGATACGGCGACCACCGAGATCTacactctttccctacacgacgctcttccgatctGCGT tcaaaataagccacTTGTTGTTGTTCTCTG and a tagA-LE bottom strand (SEQ ID NO: 18): 5′ Phospho cCAGAGAACAACAACAAgtggcttattttgaACGCagatcggaagagcgt cgtgtagggaaagagtgtAGATCTCGGTGGTCGCCGTATCATT. For SEQ ID NO: 17 the Illumina/arbitrary tag A is shown in uppercase while the sequencing priming region is shown in lower case with the 4 by barcode in uppercase followed by a 30 by Hermes Transposon end with the minimal 17 bp end shown in lower and uppercase. For SEQ ID NO: 18 the 30 by Hermes Transposon end with the minimal 17 bp end is shown in uppercase and lowercase with the 4 by barcode in uppercase followed by the sequencing priming region in lowercase and the Illumina/arbitrary tag A in uppercase.

A Hermes L-end oligo (tagB-LE) with Illumina/arbitrary tag A sequencing priming region, 4 by barcode and 30 by Hermes Transposon end is prepared by annealing tagB-LE top strand (SEQ ID NO: 19):

CAAG CAGAAGACGGCATACGAGCTCacactctttccctacacgacgctcttccgatctGCGT tcaaaataagccacTTGTTGTTGTTCTCTG and
tag B-LE bottom strand (SEQ ID NO: 20):
cCAGAGAACAACAACAAgtggcttattttgaACGCagatcggaagagcgtcgtgtagggaaagagtgtGAG CTCGTATGCCGTCTTCTGCTTG. For SEQ ID NO: 19 the Illumina/arbitrary tag B is shown in uppercase, the sequencing priming region is shown in lower case followed by a 4 by barcode in uppercase and a 30 by Hermes Transposon end with the minimal 17 bp end shown in lowercase and uppercase. For SEQ ID NO: 20 the 30 by Hermes Transposon end with the minimal 17 bp end is shown in lowercase and uppercase followed by a 4 by barcode in uppercase and a sequencing priming region and Illumina/arbitrary tag B in uppercase.

Arbitrary tags or specific Next gen sequencing platform specific tags can also be added onto the target DNA fragments by a modified method that does not need “suppression PCR” but provides a second distinct priming site using any “4-bp cutter”-restriction enzyme and a linker ligation mediated PCR approach.

In this method as shown in FIG. 9, the fragments attached to the biotinylated transferred strand are bound to magnetic Streptavidin coupled Dynal beads (Invitrogen) in binding and washing buffer (B & W buffer: 100 mM Tris-HCl, pH 8.0, 1 mM EDTA, and 1 M NaCl). The B & W buffer is removed after magnetic separation and the beads resuspended in a digestion mix that contains a restriction enzyme e.g. Msel that cuts at TTAA (NEB). Basically, a variety of affinity purification systems are adaptable to this and related methods. Various types of ligand-binding molecule systems are usable as well. Most often the small ligand is attached to the transposon and the binding molecule (receptor) is attached to a solid phase. In the illustrated examples the solid phase is composed of magnetic beads, but the solid phase can also be beads or solids in a chromatographic column or solid surfaces on a chip, etc. Biotin-Streptavidin and polyhistidine (more than six histidine residues)-nickel/cobalt binding moieties are illustrated. Lectin-sugar and hapten-antibody systems as well as other affinity systems can be used.

The bound DNA is digested at 37° C. overnight. The beads are washed and Mse1-specific linkers (obtained by annealing Linker/adapter Top strand (SEQ ID NO: 13) and Linker/adapter bottom strand (SEQ ID NO: 14) are ligated to the Mse1-digested ends of the Hermes L-end attached DNA. The beads are washed to remove non-ligated linkers. The DNA bound to the beads are used as a template for the PCR amplification of the Hermes L-end insertion site junctions using the 5′ transposon end specific primer, that has i) 5′ Illumina tag sequence fused to ii) an Illumina proprietary sequence (sequencing primer), 4-bp barcode and the Hermes Lend complementary sequence (SEQ ID NO: 15) and the 3′ linker/adapter specific primer, that has the 3′ Illumina tag (SEQ ID NO: 16). The PCR mix is separated from the Dynal beads, concentrated, the amplicons size-selected on an agarose gel and purified by gel extraction. Massively parallel sequencing is then carried out on the illumina Hi-Seq HTS platform.

The linker/adapter Top strand is SEQ ID NO: 13: TAGTCCCTTAAGCGGAGCCCTATAGTGAGTCGTATTAC. The linker/adapter bottom strand is SEQ ID NO: 14: GTAATACGACTCACTATAGGGCTCCGCTTAAGGGAC. The 5′ Transposon end specific primer is SEQ ID NO: 15: AATGATACGGCGACCACCGAGATCTacactctttccctacacgacgctcttccgatctGCGTcgcataag tatcaaaataagccac. The 3′ linker/adapter specific primer is SEQ ID NO: 16: CAAG CAGAAGACGGCATACGAGCTCttccgatctgtaatacgactcactatagggc.

For SEQ ID NO: 15 the Illumina tag A and the 4 by barcode are in uppercase while the sequencing priming region and inverted repeat are in lowercase. For SEQ ID NO: 15 the Illumina tag B is in uppercase while the linker adapter PCR priming region is in lower case.

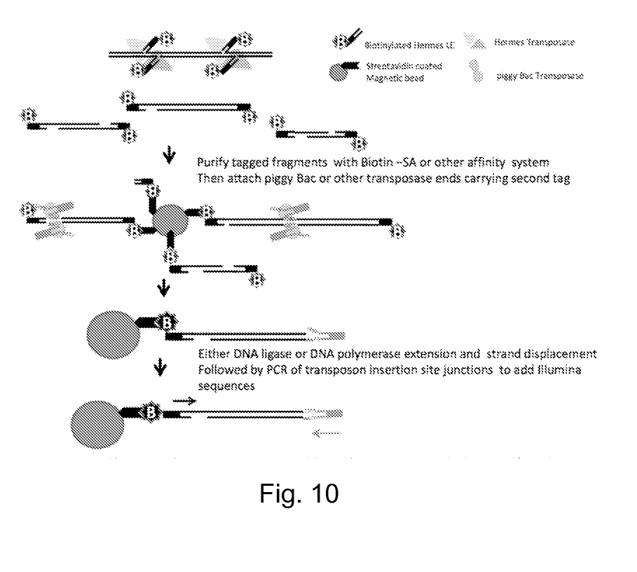

In another variation of the above embodiment (shown in FIG. 10), after tagging the 5′ ends of the target genomic DNA by strand transfer with biotinylated Hermes transposon end, instead of restriction digestion and linker ligation, a second transposase is used to provide the second tag (with a priming site distinct from the priming site provided by the Hermes transposon end) after capturing the fragments on magnetic beads. The second transposon may preferably be the piggy Bac transposase that is disclosed in and covered by Published Patent Applications US 2010/0287633, US 2010/0154070, and US 2007/0204356 (which are incorporated herein by reference to the extent allowed by applicable statute or regulation). However, any other transposase that has target DNA recognition characteristics distinct from Hermes such as SPIN, AeBuster, or even Mu and Tn5 (Nextera) can be used. This step is followed by DNA polymerase mediated extension and strand displacement using T4 DNA polymerase or DNA ligation using T4 ligase followed by PCR using primers carrying Next Gen sequencing primers.

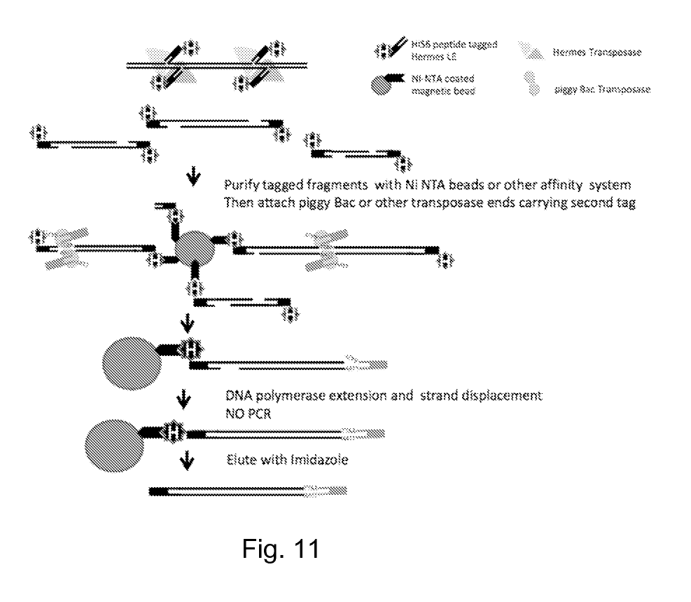

Yet another variation (shown in FIG. 11) of the above methods involves using an affinity tag, for example HIS6 (polyhistidine) peptide, covalently linked to the top strand of the transferred transposon end so that PCR amplified DNA is to be avoided prior to sequencing. In this method the DNA fragments with 8 bp single strand gaps after being immobilized on an Ni-NTA coated magnetic bead can be filled by extension and strand displacement using T4 DNA polymerase and eluted from the column using imidazole.

The following claims are thus to be understood to include what is specifically illustrated and described above, what is conceptually equivalent, what can be obviously substituted and also what essentially incorporates the essential idea of the invention. Those skilled in the art will appreciate that various adaptations and modifications of the just-described preferred embodiment can be configured without departing from the scope of the invention. The illustrated embodiment has been set forth only for the purposes of example and that should not be taken as limiting the invention. Therefore, it is to be understood that, within the scope of the appended claims, the invention may be practiced other than as specifically described herein.

References. The following references are provided to aid in understanding the invention and are incorporated herein by reference to the extent permitted by applicable statute and regulation
1. Biery M. C., Stewart F. J., Stellwagen A. E., Raleigh E. A., Craig N. L., “A simple in vitro Tn7-based transposition system with low target site selectivity for genome and gene analysis”. Nucleic Acids Res. 2000 Mar 1, 28(5):1067-77.
2. Goryshin I. Y., Reznikoff W. S., “Tn5 in vitro transposition”. ,J Biol Chem. 1998 Mar 27;273(13):7367-74
3. Haapa S, Suomalainen S, Eerikäinen S, Airaksinen M, Paulin L, Savilahti H. “An efficient DNA sequencing strategy based on the bacteriophage mu in vitro DNA transposition reaction.” Genome Res. 1999 Mar, 9(3):308-15
4. O'Brochta D. A., Warren W. D., Saville K. J., Atkinson P. W., “Hermes, a functional non-Drosophilid insect gene vector from Musca domestica”. Genetics. 1996 Mar; 142(3):907-14
5. Zhou L, Mitra R, Atkinson P. W., Hickman A. B., Dyda F, Craig N. L., “Transposition of hAT elements links transposable elements and V(D)J recombination”.Nature. 2004 Dec 23;432(7020):995-1001.
6. Perez Z. N., Musingarimi P, Craig N. L., Dyda F, Hickman A. B., “Purification, crystallization and preliminary crystallographic analysis of the Hermes transposase.” Acta Crystallogr Sect F Struct Biol Cryst Commun. 2005 Jun 1;61(Pt 6):587-90
7. Hickman A. B., Perez Z. N., Zhou L., Musingarimi P., Ghirlando R., Hinshaw J. E., Craig N. L., Dyda F., “Molecular architecture of a eukaryotic DNA transposase.” Nat Struct Mol Biol. 2005 Aug;12(8):715-21
8. Gangadharan S., Mularoni L., Fain-Thornton J., Wheelan S. J., Craig N. L., “DNA transposon Hermes inserts into DNA in nucleosome-free regions in vivo”. Proc Natl Acad Sci U S A. 2010 Dec 21;107(51):21966-72.
9. Zhou L, Mitra R, Atkinson P. W., Hickman A. B., Dyda F, Craig N. L. “Transposition of hAT elements links transposable elements and V(D)J recombination”. Nature 2004 Dec 23;432(7020):995-1001.
10. Hickman A. B., Perez Z. N., Zhou L., Musingarimi P., Ghirlando R., Hinshaw J. E., Craig N. L., Dyda F. “Molecular architecture of a eukaryotic DNA transposase”. Nat Struct Mol Biol. 2005 Aug;12(8):715-21.

What is claimed is:

1. An improved hyperactive mutant transposase having a sequence selected from the group consisting of SEQ ID NO: 5, SEQ ID NO: 6, SEQ ID NO: 7 and SEQ ID NO: 8.

2. A method of fragmenting and tagging target DNA sequences comprising the steps of:
providing ligand labeled Hermes LEs;
reacting the labeled Hermes LEs with an improved mutant transposase of claim 1 and target DNA sequences whereby each target DNA sequence becomes fragmented and each DNA fragment is labeled at either end by one of the labeled Hermes LEs;
purifying the labeled DNA fragments using an affinity system that binds the ligand.

3. The method according to claim 2, wherein the affinity system employs beads that bind the ligand.

4. The method according to claim 3, wherein the beads are magnetic beads.

5. The method according to claim 2 further comprising a step of using a DNA polymerase to fill in gaps.

6. The method according to claim 5, wherein the wherein the DNA polymerase is T4 polymerase.

7. The method according to claim 2, wherein the ligand is biotin or polyhistidine of at least six histidine residues and the affinity system is biotin-streptavidin or nickel or cobalt affinity material, respectively.

8. The method according to claim 2 further comprising a step of enzymatically cutting the tagged DNA following the step of purifying to replace one of the labeled Hermes LEs on each fragment with a specific terminal sequence.

9. The method according to claim 8, wherein PCR, DNA ligase or DNA polymerase chain extension is used to add the specific terminal sequence.

10. The method according to claim 2 further comprising the step of using a second transposon system to introduce a second tag into each DNA fragment.

11. The method according to claim 10, wherein the step of using a second transposon system follows the step of purifying.

12. The method according to claim 10, wherein the second transposon system is a piggy Bac transposon.

13. A method of fragmenting and tagging target DNA sequences comprising the steps of:
providing tagged Hermes LEs bearing at least one specific sequence tag; and
reacting the tagged Hermes LEs with an improved mutant transposase of claim 1 and target DNA sequences whereby each target DNA sequence becomes fragmented and each DNA fragment is labeled at either end by one of the tagged Hermes LEs.

14. The method according to claim 13 further comprising a step of employing a DNA polymerase to fill in gaps.

15. The method according to claim 14, wherein the DNA polymerase is T4 polymerase.

16. The method according to claim 13 further comprising the step of using a second transposon system to introduce a second tag into each DNA fragment.

17. The method according to claim 16, wherein the second transposon system is a piggy Bac transposon.

18. The method according to claim 13 further comprising a step of enzymatically cutting the tagged DNA following the step of purifying to replace one of the tagged Hermes LEs on each fragment with a specific terminal sequence.

19. The method according to claim 18, wherein PCR, DNA ligase or DNA polymerase chain extension is used to add the specific terminal sequence.
